(19) United States
(12) Patent Application Publication
(10) Pub. No.: US 2006/0116319 A1
(43) Pub. Date: Jun. 1, 2006

(54) Method for identifying and treating photodamaged skin

(75) Inventors: Susanne Teklits Iobst, Maywood, NJ (US); Kurt Matthew Schilling, North Caldwell, NJ (US); Charles David Boyd, Honolulu, HI (US); Johann Georg Eugen Urschitz, Honolulu, HI (US); Gordon S. Okimoto, Honolulu, HI (US)

Correspondence Address: UNILEVER INTELLECTUAL PROPERTY GROUP, 700 SYLVAN AVENUE,, BLDG C2 SOUTH, ENGLEWOOD CLIFFS, NJ, 07632-3100, US

(73) Assignee: Unilever Home & Personal Care USA, Division Of Conopco, Inc.

(21) Appl. No.: 10/998,685

(22) Filed: Nov. 29, 2004

(51) Int. Cl. A61K 48/00; A61K 38/17

(52) U.S. Cl. 514/12; 514/44; 424/59

(57) ABSTRACT

The present invention relates to modulation of MAP2K2 and DUSP-1 genes and proteins encoded thereby. More specifically, the present invention relates to modulation of MAP2K2 and DUSP-1 genes to detect and condition photo-damaged skin, preferably, chronically photo-damaged skin, as well as to identify active compounds and compositions for skin conditioning.

### FIELD OF THE INVENTION

The present invention relates to modulation of MAP2K2 and DUSP-1 genes and proteins encoded thereby, which can be used as markers of photo-damage in skin. More specifically, the present invention relates to modulation of MAP2K2 and D USP-1 for identifying and conditioning chronic sun-exposure in moderately photo-damaged skin.

### BACKGROUND OF THE INVENTION

All the genes of a cell comprise the genome. The human genome contains approximately 100,000 genes. However, in any given cell, only a fraction of these genes is expressed. Each gene is expressed at a precise time and at a precise level.

A typical mammalian cell of a given lineage expresses approximately 20,000-30,000 of the 100,000 odd germ line genes carried in its genome. Almost all cells universally express many of the genes, which are called “housekeeping” genes. Examples of housekeeping genes include genes encoding enzymes involved in glycolysis or proteins involved in cell structure. However, it is the non-universally expressed genes that differentiate cells from each other. As cells mature into differentiated cells, certain non-constitutively expressed genes are turned on and off at different stages. Thus, the differences in gene expression patterns between cells make, for example, a nerve cell different from a blood cell.

Under abnormal cellular conditions such as those in individuals with disease or disorders, the pattern of gene expression within individual cells may be changed compared to the expression pattern seen under normal non-disease conditions. A change in gene expression may be an effect or the cause of a disease or abnormality, such as in, for example, a tumour cell. Whereas some diseases may be understood as caused by mutations in particular genes and thus potentially be detected by examining the genomic sequence, many diseases and disorders involve a malfunction in the level of expression of genes which cannot be detected by sequencing the genome but can only be detected by identifying the gene expression patterns of the cells. Therefore, in order to understand the function of specific cell types in an organism or to understand the progression of a disease or disorder, it is necessary to understand the expression status of individual genes within these specific cell types at different stages of the organism's development.

Researchers have therefore used many techniques such as isolating proteins from various cells and comparing the abundance of each of the proteins. Another method involves the use of antibodies to probe populations of peptides produced from mRNA pools. Therefore, “libraries” of synthetic polypeptides corresponding to the polypeptides coded for by mRNA molecules are produced and then probed by individual antibodies, as described in U.S. Pat. No. 5,242,798.

The present invention relates to genes that function as markers of photdamage and the modulation thereof to detect and treat photodamaged skin.

Intrinsic, environmental, and lifestyle factors all contribute to the process of skin aging. Aging of the skin is thought to consist of two processes taking place simultaneously. The first process is intrinsic, chronologic aging and similar perhaps to aging of other tissues. The second process is photoaging, an environmentally-induced remodeling of the dermis that arises as a result of repeated exposure of skin to sunlight (UV-Light). Although recent studies (e.g., Varani, J., R. L. Warner, et al. , “Vitamin A antagonizes decreased cell growth and elevated collagen-degrading matrix metalloproteinases and stimulates collagen accumulation in naturally aged human skin.” J Invest Dermatol 114(3): 480-6 (2000)), have shown that both intrinsic aging and photoaging share some common characteristics, such as decreased procollagen gene expression and increased expression of genes encoding several matrix metalloproteinases, it has been suggested that photoaging is the predominant contributing factor to the prematurely aged appearance of sun-exposed skin. The histopathological features of photoaging are mostly alterations in a variety of extracellular matrix (ECM) proteins such as degradation of collagen, increased g lycosaminoglycan content and a massive accumulation of elastotic material in the superficial dermis (solar elastosis).

Few studies have addressed the long-term biosynthetic consequences of chronic and repeated sun exposure. Fisher, G. J., Z. Q. Wang, et al. “Pathophysiology of premature skin aging induced by ultraviolet light,” N Enql J Med 337(20): 1419-28 (1997), for example, have proposed the model of UV-induced, MAP-kinase (mitogen-activated protein kinase) mediated activation of matrix metalloproteinases as the underlying mechanism for the aberrant remodeling of collagens as a direct consequence of repeated UV exposure. Most of the studies in support of this hypothesis however, have been conducted using UV-irradiated sun-protected skin. Chung et al., “Decreased Extracellular-Signal-Regulated Kinase and Increased Stress-Activated MAP Kinase Activities in Aged Human Skin In Vivo,” J. Invest. Dermatol., 114: 177-182 (2000) make a study of young versus old skin. However, the study on which the present invention is based was designed to assess constitutive changes in gene expression as a consequence of decades of chronic sun exposure relative to matched sun-protected skin.

### SUMMARY OF THE INVENTION

The present invention relates to a personal care method of modulating (inhibit downregulation or upregulate) MAP2K2 and/or DUSP1 genes, including proteins encoded thereby, in skin of an individual comprising applying to skin an active ingredient for conditioning of skin. Skin condition is selected from the group consisting of photo-damage, aging, appearance of wrinkles, age spots, stratum corneum flexibility, dry skin, acne, oily skin, general quality of skin, skin desquamation and epidermal differentiation. The personal care method is particular useful for conditioning skin photo-damage.

In another aspect, the present invention relates to a personal care method of conditioning photo-damaged skin comprising inhibiting downregulation or upregulating MAP2K2 and /or DUSP-1 gene by topically applying to the skin an active ingredient therefor.

In another aspect, the present invention relates to a personal care method of identifying active ingredients for skin conditioning comprising:
a) using MAP2K2 and/or DUSP-1 gene as a marker of photo-damage in skin;
b) applying an ingredient to the skin of an individual or to epidermal cells; and
c) determining a change in the marker to determine efficacy of the ingredient for treating photo-damage.
The change in the marker may be referred to as modulation, including inhibiting downregulation or upregulating MAP2K2 and /or DUSP-1 gene in the skin.

In another aspect, the present invention relates to a personal care skin conditioning composition comprising:
a) an active ingredient identified using the inventive method of using MAP2K2 and/or DUSP1 genes, or proteins encoded thereby, as markers of photodamage; and
b) a cosmetically acceptable vehicle.

In a further aspect, the present invention relates to a personal care method for detecting a photo-damaged skin condition in an epidermal sample, the method comprising the steps of:

a) using MAP2K2 and/or DUSP-1 gene as a marker of photo-damage; and

b) determining a change in the marker to determine presence of photo-damage.

### DETAILED DESCRIPTION

The present invention relates to a method for detecting and/or conditioning, particularly the condition of photodamage, in the epidermis of an individual using MAP2K2 and/or DUSP-1 as markers of the skin condition.

As used herein, the term “comprising” means including, made up of, composed of, consisting and/or consisting essentially of. Furthermore, in the ordinary meaning of “comprising,” the term is defined as not being exhaustive of the steps, components, ingredients, or features to which it refers.

Except in the operating and comparative examples, or where otherwise explicitly indicated, all numbers in this description indicating amounts or ratios of material or conditions of reaction, physical properties of materials and/or use are to be understood as modified by the word “about”.

The term “conditioning” as used herein means improvement and/or treatment of dry skin, acne, photo-damaged skin, appearance of wrinkles, age spots, aged skin, increasing stratum corneum flexibility, lightening skin color, controlling sebum secretion and generally increasing the quality of skin. The term includes improving skin desquamation and epidermal differentiation. The term also includes reversing the aged appearance of sun-damaged skin.

The term “epidermis” refers to the outer, nonvascular, layer of the skin that overlies the dermis.

The term “skin” as used herein includes the skin on or in the face, mouth, neck, chest, back, arms, hands, legs, and scalp. The term “epidermis” is viewed as being encompassed by the term “skin.”

“Medical Applications” are devices and compositions which are distributed solely by prescription or solely to the medical profession.

“Personal Care Applications” are devices and compositions for the cleaning and care of human skin, except Medical Applications.

A “gene” is a unit of inheritable genetic material found in a human chromosome. The recurring structural units of all nucleic acids are eight different nucleotides; four kinds of nucleotides are the building blocks of DNA, and four others are the structural units of RNA. For example, the four-letter language of DNA is translated into the twenty-letter language of protein.

The term “photo-damage” refers to photo-aging as discussed above with relation to skin. The term includes but is not limited to skin aging as a consequence of decades of chronic sun exposure.

Gene expression analysis is a tool that can be utilized to identify those markers that are indicative of specific skin conditions such as photo-damage or dry skin. Photo-damage is not well understood biologically. The present invention provides a method for identifying skin conditions by using markers such as genes which are indicative of a particular skin condition. Specifically, the present invention provides a method of using MAP2K2 and/or DUSP-1 genes that are modulated in skin conditions and therefore can be used as markers of skin conditions.

In one aspect, the present invention relates to a personal care method of modulating MAP2K2 and/or DUSP1 gene (inhibiting downregulation or upregulating) by applying to the skin of an individual an active ingredient for improvement of skin condition. In a preferred aspect, the method is directed to the skin condition of photo-damage.

In another aspect, the present invention relates to a personal care method of conditioning photo-damaged skin comprising inhibiting downregulation or upregulating MAP2K2 and /or DUSP-1 genes by topically applying to the skin of an individual an active ingredient therefor.

In a further aspect, the present invention relates to a personal care method of identifying active ingredients for improvement of skin condition comprising:
c) using MAP2K2 and/or DUSP-1 gene as a marker of photo-damage in skin;
d) applying an ingredient to the skin of an individual or to epidermal cells; and
c) determining a change in the marker to determine efficacy of the ingredient for treating photo-damage.
The change in the marker may be referred to as modulation, including inhibiting downregulation or upregulating MAP2K2 and /or DUSP-1 gene in the skin.

In still a further aspect, the present invention relates to a personal care skin conditioning composition comprising:
a) an active ingredient identified using the inventive method of using MAP2K2 and DUSP1 genes, or proteins encoded thereby, as markers of photo-damage; and
b) a cosmetically acceptable vehicle.

In another embodiment, the present invention comprises a personal care method for detecting a photo-damaged skin condition in an epidermal sample comprising the steps of:
a) using MAP2K2 and/or DUSP-1 genes, or proteins encoded thereby, as markers of photo-damage; and
b) determining a change in the marker to determine presence of photo-damage.
Applicants have discovered that the change in the marker is downregulation when the epidermal sample is that of chronically sun-damaged skin.
The method comprises a first step of selecting a first skin sample that is known not to be photo-damaged. Next is selecting a second skin sample the condition of which is to be identified. The first sample is compared to the second sample to determine whether there is a change in the gene or the amount of the gene, or the protein encoded thereby. If there is a change in the gene, specifically, downregulation, then photo-damage exists in the second sample.

MAP2K2: mitogen-Activated Protein Kinase Kinase 2

MAP2K2 has GenBank Accession No. L11285. The protein encoded by MAP2K2 is a dual specificity protein kinase that belongs to the MAP kinase kinase family. Zheng, et al., “Cloning and Characterization of Two Distinct Human Extracellular Signal-regulated Kinase Activator Kinases, MEK1 and MEK2,” The Journal of Biological Chemistry, 268:11435-11439 (1993). This kinase plays a critical role in mitogen growth factor signal transduction. It phosphorylates and thus activates MAPK1/ERK2 and MAPK2/ERK3. The activation of this kinase itself is dependent on the Ser/Thr phosphorylation of MAP kinase kinase kinases.

ERK2 is an extracellular signal-regulated kinase 2, having GenBank accession No. M84489. ERK3 is an extracellular signal-regulated kinase 3, having GenBank accession No. X80692.

DUSP1: Dual Specificity Phosphatase 1

The present invention is directed to the DUSP1 gene, GenBank Accession No. X68277, as a second member of the MAPK signaling pathway. DUSP1 is a dual specificity phosphatase 1 (also called MKP1).

The expression of the DUSP1 gene is induced in human skin fibroblasts by oxidative/heat stress and growth factors. Farooq, A. and M. M. Zhou, “Structure and regulation of MAPK phosphatases,” Cell Signal 16(7): 769-79 (2004). The DUSP1 gene specifies a protein with structural features similar to members of the non-receptor-type protein-tyrosine phosphatase family and has significant amino-acid sequence similarity to a Tyr/Ser-protein phosphatase encoded by the gene H1 of vaccinia virus. The bacterially expressed and purified DUSP1 protein has intrinsic phosphatase activity and specifically inactivates mitogen-activated protein (MAP) kinase in vitro by the concomitant dephosphorylation of both its phospho-threonine and phospho-tyrosinase residues. Furthermore, it suppresses the activation of MAP kinase by oncogenic ras in extracts of Xenopus oocytes. Thus, it was thought DUSP1 may play an important role in human cellular response to environmental stress as well in the negative regulation of cellular proliferation.

The following specific examples further illustrate the invention, but the invention is not limited thereto.

### EXAMPLES

Applicants have investigated constitutive changes in gene expression arising as a result of chronic exposure to sunlight (as opposed to methods studying changes arising as a consequence of recent, acute UV irradiation). Comparative studies were undertaken of sun-damaged pre-auricular skin and sun-protected post-auricular skin.

The following methods were employed in the examples.

To determine the inter-individual differences in gene expression profiles, Applicants used total RNA preparations from skin obtained from 11 Caucasian patients with moderate sun damage and analyzed the changes in mRNA levels encoded by more than 22,000 different genes represented on the Affymetrix U133A chip. Applicants developed an algorithm that exploits dependencies between control and test chips by computing a paired t-statistic for each gene as a measure of differential expression and a false discovery rate for lists of genes called significantly changed based on permutation testing. Using this algorithm, Applicants were able to identify 566 mRNAs with consistently altered steady-state levels as a consequence of chronic sun exposure.
Extraction of mRNA. Total RNA from pre- and post-auricular skin samples were extracted using RNA STAT-60 (Tel-Test, Friendswood, Tex.) following the manufacturer's protocol. DNA was removed with on-column DNase digestion using a Qiagen RNases-free DNase set. RNA samples were stored in water at −80° C. until hybridization. The RNA was quantified by spectrophotometry (Beckman, Fullerton, Calif.). The integrity Poly(A⁺)RNA was confirmed by electrophoresis through 1.2% agarose gels containing formaldehyde.
Array preparation: Fifty micro-g of total RNA was reverse-transcribed, amplified, and labeled as described (Mahadevappa, M., et al., “A High-Density Probe Array Sample Preparation Method Using 10- to 100-fold Fewer Cells,” Nat. Biotechnol., 17(11):1134-6 (1999); Le, X. F., et al, “Genes Affecting the Cell Cycle, Growth, Maintenance, and Drug Sensistivity Are Preferrentially Regulated By Anti-HER2 antibody through PI3K-AKT signaling,” J. Biol. Chem., (2001)). Labeled cRNA was hybridized to HGU133A arrays (Affymetrix) with the capacity to display transcript levels of 22,283 human genes. Arrays were washed, stained with antibiotin streptavidin-phycoerythrin-labeled antibody using Affymetrix fluidics station and then scanned using the Affymetrix GeneArray scanner system.

Using the micro-array technique, Applicants have found that MAP2K2 and DUSP-1 are down-regulated in photo-damaged skin. To confirm the findings, as shown in the Examples below, Applicants have determined how modulation of MAP2K2 and DUSP1 correlates to differences in protein levels.

### Example 1

This Example illustrates the differences in steady-state mRNA levels in pre-auricular skin as compared with post-auricular skin by Quantitative RT-PCR.

Applicants conducted a quantitative RT-PCR analysis for two different RNAs, MAP2K2 and DUSP1.
Quantitative RT-PCR: PCR primers (IDT DNA, xxx.) and probes (PE Biosystems, Foster City, Calif.) for xx were designed using the computer program Primer Express (PE Biosystems). Primers used for amplification were 5′-GGCGTAAGAAACGCTT TGCA-3′ (forward, nucleotides 765-784) and 5′-CAGTTCACCAG GCGGTAGGA-3′ (reverse, nucleotides 834-816). Fluorogenic probes contained a reporter dye (FAM) covalently attached at the 5′ end and a quencher dye (TAMRA) covalently attached at the 3′ end.

RNA samples from 11 individuals were analyzed. The mean threshold cycle (c(t)) number from three independent PCR reactions was used to calculate the number of MAP2K2 or DUSP1 RNA copies added to each reaction. The results of this analysis are depicted in the Table below.

While there is inter-patient variation in the level of down-regulation, the overall result for both enzymes demonstrates a clear trend to reduced RNA levels in pre-auricular skin, i.e. photo-damaged skin.

TABLE 1
Quantitative RT-PCR
MAP2K2

| Subject | Sample | C(T)* | Copy #* |
|---|---|---|---|
| Subject 1 | Pre | 26.21 | 1.37E + 07 |
| | Post | 23.72 | 7.72 E + 07 |
| Subject 2 | Pre | 24.43 | 4.71E + 07 |
| | Post | 23.64 | 8.28E + 07 |
| Subject 3 | Pre | 25.53 | 2.16E + 07 |
| | Post | 25.43 | 3.11E + 07 |
| Subject 4 | Pre | 26.61 | 1.04E + 07 |
| | Post | 26.25 | 1.30E + 07 |
| Subject 5 | Pre | 28.77 | 2.15E + 06 |
| | Post | 27.04 | 7.43E + 06 |
| Subject 6 | Pre | 26.67 | 9.54E + 06 |
| | Post | 25.93 | 1.63E + 07 |
| Subject 7 | Pre | 26.42 | 1.14E + 07 |
| | Post | 26.43 | 1.14E + 07 |
| Subject 8 | Pre | 27.50 | 5.32E + 06 |
| | Post | 28.20 | 3.30E + 06 |
| Subject 9 | Pre | 28.01 | 3.71E + 06 |
| | Post | 27.48 | 5.47E + 06 |
| Subject 10 | Pre | 28.58 | 2.98E + 06 |
| | Post | 28.71 | 5.87E + 06 |
| Subject 11 | Pre | 29.22 | 1.57E + 06 |
| | Post | 27.31 | 7.00E + 06 |

TABLE 1A
MAP2K2

| | Fold Change |
|---|---|
| Subject | Pre-Auricular Skin |
| 1 | −5.5 |
| 2 | −1.8 |
| 3 | −1.4 |
| 4 | −1.1 |
| 5 | −3.4 |
| 6 | −1.7 |
| 7 | −1.0 |
| 8 | −0.8 |
| 9 | −1.5 |
| 10 | −1.9 |
| 11 | −4.5 |

TABLE 2
DUSP1

| Subject | Sample | C(T)* | Copy#* |
|---|---|---|---|
| Subject 1 | Pre | 24.103 | 9.6E + 08 |
| | Post | 21.380 | 6.8E + 09 |
| Subject 2 | Pre | 21.287 | 7.3E + 09 |
| | Post | 20.213 | 1.6E + 10 |
| Subject 3 | Pre | 23.186 | 1.8E + 09 |
| | Post | 22.197 | 3.8E + 09 |
| Subject 4 | Pre | 24.130 | 9.2E + 08 |
| | Post | 23.382 | 1.6E + 09 |
| Subject 5 | Pre | 27.839 | 5.8E + 07 |
| | Post | 25.218 | 4.0E + 08 |
| Subject 6 | Pre | 22.919 | 2.2E + 09 |
| | Post | 22.615 | 2.8E + 09 |
| Subject 7 | Pre | 25.072 | 4.5E + 08 |
| | Post | 24.342 | 8.4E + 08 |
| Subject 8 | Pre | 25.252 | 3.9E + 08 |
| | Post | 25.299 | 3.8E + 08 |
| Subject 9 | Pre | 24.848 | 5.5E + 08 |
| | Post | 25.461 | 3.4E + 08 |
| Subject 10 | Pre | 27.199 | 9.2E + 07 |
| | Post | 24.484 | 7.0E + 08 |
| Subject 11 | Pre | 26.017 | 2.2E + 08 |
| | Post | 26.014 | 2.2E + 08 |

*Average of 3

TABLE 2A
DUSP1

| | Fold Change |
|---|---|
| Subject | Pre-Auricular Skin |
| 1 | −7.1 |
| 2 | −2.2 |
| 3 | −2.1 |
| 4 | −1.9 |
| 5 | −6.8 |
| 6 | −1.3 |
| 7 | −1.9 |
| 8 | −1.0 |
| 9 | −0.8 |
| 10 | −7.6 |
| 11 | −1.0 |

### Example 2

This Example demonstrates that protein levels of MAP2K2 are decreased in sun-damaged pre-auricular skin, as detected by Western Blot analysis.

To confirm that the mRNA levels observed in the microarray analysis were consistent with protein expression, MAP2K2 was detected by Western Blot analysis. Proteins were isolated (protein extracts, 30 micro-g each) from pre- and post-auricular skin samples from 7 female Caucasian subjects in their fifth decade of life with moderate sun damage. Total RNA from Subjects 1-4 of these 7 had been used for the transcriptional profiling of photo-aged skin described above whereas the samples from the other 3 subjects had not been used previously. Polyclonal rabbit antibody was used to detect MAP2K2.

In the Table below, MAP2K2 levels of the 7 subjects are depicted as ratio of pre-and post-auricular skin. The results from this Western blot analysis clearly demonstrate that protein levels of MAP2K2 are decreased in the sun-damaged pre-auricular skin of 6 of the 7 investigated samples. Only subject 7 showed an increased MAP2K2 protein level in pre-auricular skin.

TABLE 3
Fold Change
Pre-Auricular

| Subject | Skin |
|---|---|
| 1 | −3.1% |
| 2 | −4.3% |
| 3 | −1.8% |
| 4 | −1.7% |
| 5 | −2.3% |
| 6 | −3.4% |
| 7 | −2.1% |

### Example 3

This Example illustrates the decreased levels of MAP2K2 and DUSP1 in photodamaged skin using Immunohistochemical Analysis.
Immunohistochemistry: Immunostaining was performed by the avidin-biotin-peroxidase technique (Vectastain ABC Kit, Vector Laboratories, Burlingame, Calif.) as described previously (Dahlback, et al., “Fibrillin Immunoreactive fibers Constitute a Unique Network in the Human Dermis: Immunohistochemical comparison of the Distributions of Fibrillin, Vitronectin, Amylioid P Component, and Orcein Stainable Structures in Normal Skin and Elastosis,” J. Invest. Dermatol., 94(3):284-91 (1990)) using pretested dilutions of affinity-purified XX rabbit antibodies. Diaminobenzidine or aminoethylcarbazole were used as chromogenic substrates and Harris hematoxylin as counterstain. The sections were pretreated by antigen retrieval as described previously (Grabenbauer, et al., 2001) and endogenous peroxidase was inactivated by 0.3% H₂0₂ for 30 min. MEK-2 was detected using a polyclonal antibody (1:200; Santa Cruz Biotechnology, California). For negative controls, sections with pre-immune serum were processed, or affinity-purified antibodies were incubated with excess peptide antigen to inhibit specific interactions with XX in the tissue.

a. Detection of MAP2K2 Protein in Normal and Photoaged Skin

Paired biopsies of photoaged pre-auricular skin and sun-protected post-auricular skin obtained from 6 patients were used to study the changes in protein levels associated with photoaging. RNA of pre-and post-auricular skin from all 6 subjects had been used for the microarray analysis. Five micron full-thickness paraffin-embedded skin sections of pre-and post-auricular skin were stained for MAP2K2 using purified polyclonal rabbit anti-human MAP2K2 antibody. For the negative control slide, the primary antibody was replaced with 1% normal goat serum.

Consistent with the Western blot analysis results shown above, the immunohistochemical analysis of MAP2K2 revealed decreased protein levels in the epidermis of the pre-auricular slides. Expression of this protein could be detected in the cytoplasm throughout all epidermal layers. A quantitative analysis of the epidermal area positively stained for MAP2K2 protein in pre- and post-auricular skin using the image analysis software IMAGE Pro Plus confirmed a statistically significant decrease (p<0.022) of MAP2K2 in pre-auricular skin sections of all 6 subjects.

b. Detection of DUSP1 Protein in Pre- and Post-Auricular Skin

Paired biopsies of sun-damaged pre-auricular skin and sun-protected post-auricular skin obtained from 6 patients were used to study the changes in DUSP1 protein expression. Sections of pre-and post-auricular skin were stained for DUSP1 using purified polyclonal rabbit anti-human DUSP1 antibody. For the negative control, the primary antibody was replaced with 1 % normal goat serum.

While the changes in DUSP1 protein expression were not as marked as for MAP2K2, higher levels of DUSP1 protein were evident in post-auricular skin. Positive immuno-staining could be detected in cytoplasm of all cell layers of the epidermis. The stratum corneum displayed strong staining at similar levels for both pre- and post-auricular samples.

Using IMAGE Pro Plus brand computer program, the area of DUSP1 positive staining in the skin sections of pre- and post-auricular skin were quantified. This analysis revealed statistically significant (p<0.036) decrease in the area positively stained for DUSP1 in all 6 subjects (see Table below).

TABLE 4A
Percent Area in the
Epidermis by Subject

| | |
|---|---|
| F3 | 60% |
| P7 | 32% |
| P8 | 37% |
| R11 | 41% |
| R13 | 78% |
| R2 | 58% |

TABLE 4B
Percent Area Positively
Stained in the Epidermis

| | |
|---|---|
| Post | 79% |
| MAP2K2 | |
| Pre | 31% |
| MAP2K2 | |
| Post | 61% |
| DUSP1 | |
| Pre | 33% |
| DUSP1 | |

Table 4 summarizes an analysis of the Percent area positively stained in the epidermis. Table 4A shows the epidermal area positively stained for each subject, while, in Table 4B, the average area positively stained over all 6 subjects is displayed.

The levels for both proteins showed a statistically significant decrease in staining in the epidermis of the pre-auricular skin sections (* p<0.022, ** p<0.036).

Taken together, Applicants have provided evidence that two members of the ERK kinase (extra-cellular signal-regulated kinase) pathway show lower steady-state mRNA levels as well as less activated protein levels in sun-damaged pre-auricular skin. Lower pre-auricular levels for MAP2K2 and DUSP1 could be identified by quantitative RT-PCR and immuno-histochemistry. Originally, DUSP1 was identified as a negative feedback regulator of ERK signaling. Therefore, reduced levels DUSP1 mRNA and protein levels in pre-auricular skin may be a consequence of constitutively lower ERK levels in pre-auricular skin which in turn require less of the negative feedback mechanism.

Actives and Compositions Identified Using MAP2K2 and/or DUSP1 as Targets

In a further aspect, the present invention relates to a personal care method of identifying active ingredients for improvement of skin condition comprising:
a) using MAP2K2 and/or DUSP-1 gene as a marker of photo-damage in skin;
b) applying an ingredient to the skin of an individual or to epidermal cells; and
c) detecting a change in the marker to determine efficacy of the ingredient for treating photo-damage.
The change in the marker may be referred to as modulation, including inhibiting downregulation or upregulating MAP2K2 and /or DUSP-1 gene in the skin.

In still a further aspect, the present invention relates to a personal care skin conditioning composition comprising:
a) an active ingredient identified using the inventive method of using MAP2K2 and DUSP1 genes as markers of photo-damage; and
b) a cosmetically acceptable vehicle.

The compositions according to the invention also comprise a cosmetically acceptable vehicle to act as a dilutant, dispersant or carrier for the active ingredients in the composition, so as to facilitate their distribution when the composition is applied to the skin.

The vehicle may be aqueous, anhydrous or an emulsion. Preferably, the compositions are aqueous or an emulsion, especially water-in-oil or oil-in-water emulsion, preferentially oil in water emulsion. Water when present will be in amounts which may range from 5 to 99%, preferably from 20 to 70%, optimally between 40 and 70% by weight.

Besides water, relatively volatile solvents may also serve as carriers within compositions of the present invention. Most preferred are monohydric C₁-C₃ alkanols. These include ethyl alcohol, methyl alcohol and isopropyl alcohol. The amount of monohydric alkanol may range from 1 to 70%, preferably from 10 to 50%, optimally between 15 to 40% by weight.

Emollient materials may also serve as cosmetically acceptable carriers. These may be in the form of silicone oils and synthetic esters. Amounts of the emollients may range anywhere from 0.1 to 50%, preferably between 1 and 20% by weight.

Silicone oils may be divided into the volatile and non-volatile variety. The term “volatile” as used herein refers to those materials which have a measurable vapor pressure at ambient temperature. Volatile silicone oils are preferably chosen from cyclic or linear polydimethylsiloxanes containing from 3 to 9, preferably from 4 to 5, silicon atoms. Linear volatile silicone materials generally have viscosities less than about 5 centistokes at 25° C. while cyclic materials typically have viscosities of less than about 10 centistokes. Nonvolatile silicone oils useful as an emollient material include polyalkyl siloxanes, polyalkylaryl siloxanes and polyether siloxane copolymers. The essentially non-volatile polyalkyl siloxanes useful herein include, for example, polydimethyl siloxanes with viscosities of from about 5 to a bout 25 million centistokes at 25° C. Among the preferred non-volatile emollients useful in the present compositions are the polydimethyl siloxanes having viscosities from about 10 to about 400 centistokes at 25° C.

Among the ester emollients are:
(1) Alkenyl or alkyl esters of fatty acids having 10 to 20,carbon atoms.

Examples thereof include isoarachidyl neopentanoate, isononyl isonanonoate, oleyl myristate, oleyl stearate, and oleyl oleate.
(2) Ether-esters such as fatty acid esters of ethoxylated fatty alcohols.
(3) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monooleate, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters.
(4) Wax esters such as beeswax, spermaceti, myristyl myristate, stearyl stearate and arachidyl behenate.
(5) Sterols esters, of which cholesterol fatty acid esters are examples.

Fatty acids having from 10 to 30 carbon atoms may also be included as cosmetically acceptable carriers for compositions of this invention. Illustrative of this category are pelargonic, lauric, myristic, palmitic, stearic, isostearic, hydroxystearic, oleic, linoleic, ricinoleic, arachidic, behenic and erucic acids.

Humectants of the polyhydric alcohol-type may also be employed as cosmetically acceptable carriers in compositions of this invention. The humectant aids in increasing the effectiveness of the emollient, reduces scaling, stimulates removal of built-up scale and improves skin feel. Typical polyhydric alcohols include glycerol, polyalkylene glycols and more preferably alkylene polyols and their derivatives, including propylene glycol, dipropylene glycol, polypropylene glycol, polyethylene glycol and derivatives thereof, sorbitol, hydroxypropyl sorbitol, hexylene glycol, 1,3-butylene glycol, 1,2,6-hexanetriol, ethoxylated glycerol, propoxylated glycerol and mixtures thereof. For best results the humectant is preferably propylene glycol or sodium hyaluronate. The amount of humectant may range anywhere from 0.5 to 30%, preferably between 1 and 15% by weight of the composition.

Thickeners may also be utilized as part of the cosmetically acceptable carrier of compositions according to the present invention. Typical thickeners include crosslinked acrylates (e.g. Carbopol 982), hydrophobically-modified acrylates (e.g. Carbopol 1382), cellulosic derivatives and natural gums. Among useful cellulosic derivatives are sodium carboxymethylcellulose, hydroxypropyl methylcellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Amounts of the thickener may range from 0.0001 to 5%, usually from 0.001 to 1%, optimally from 0.01 to 0.5% by weight.

Collectively the water, solvents, silicones, esters, fatty acids, humectants and/or thickeners will constitute the cosmetically acceptable carrier in amounts from 1 to 99.9%, preferably from 80 to 99% by weight.

Optional Skin Benefit Materials and Cosmetic Adjuncts:

An oil or oily material may be present, together with an emulsifier to provide either a water-in-oil emulsion or an oil-in-water emulsion, depending largely on the average hydrophilic-lipophilic balance (HLB) of the emulsifier employed.

Surfactants may also be present in cosmetic compositions of the present invention. Total concentration of the surfactant will range from 0.1 to 40%, preferably from 1 to 20%, optimally from 1 to 5% by weight of the composition. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di- fatty acid esters of ethylene glycol; fatty acid monoglyceride; sorbitan, mono- and di- C8-C₂₀ fatty acids; block copolymers (ethylene oxide/propylene oxide); and polyoxyethylene sorbitan as well as combinations thereof. Alkyl polyglycosides and saccharide fatty amides (e.g. methyl gluconamides) are also suitable nonionic surfactants.

Preferred anionic surfactants include soap, alkyl ether sulfate and sulfonates, alkyl sulfates and sulfonates, alkylbenzene sulfonates, alkyl and dialkyl sulfosuccinates, C₈-C₂₀ acyl isethionates, acyl glutamates, C₈-C₂₀ alkyl ether phosphates and combinations thereof.

Various types of additional active ingredients may be present in cosmetic compositions of the present invention. Actives are defined as skin benefit agents other than emollients and other than ingredients that merely improve the physical characteristics of the composition. Although not limited to this category, general examples include additional anti-oxidants, anti-aging ingredients and sunscreens.

Sunscreens include those materials commonly employed to block ultraviolet light. Illustrative compounds are the derivatives of PABA, cinnamate and salicylate. For example, avobenzophenone (Parsol 1789®)) octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the compositions can vary depending upon the degree of protection desired from the sun's UV radiation.

Many cosmetic compositions, especially those containing water, must be protected against the growth of potentially harmful microorganisms. Preservatives are, therefore, necessary. Suitable preservatives include alkyl esters of p-hydroxybenzoic acid, hydantoin derivatives, propionate salts, and a variety of quaternary ammonium compounds. Particularly preferred preservatives of this invention are methyl paraben, propyl paraben, phenoxyethanol and benzyl alcohol. Preservatives will usually be employed in amounts ranging from about 0.1% to 2% by weight of the composition.

Powders may be incorporated into the cosmetic composition of the invention. These powders include chalk, talc, Fullers earth, kaolin, starch, smectites clays, chemically modified magnesium aluminum silicate, organically modified montmorillonite clay, hydrated aluminum silicate, fumed silica, aluminum starch octenyl succinate and mixtures thereof.

The composition according to the invention is intended primarily as a product for topical application to human skin, especially as an agent for conditioning the skin, and is particularly useful for conditioning photo-damaged skin.

In use, a quantity of the composition, for example from 1 to 100 ml, is applied to exposed areas of the skin, from a suitable container or applicator and, if necessary, it is then spread over and/or rubbed into the skin using the hand or fingers or a suitable device.

Product Form and Packaging:

The cosmetic skin composition of the invention can be in any form, e.g. formulated as a gel, lotion, a fluid cream, or a cream. The composition can be packaged in a suitable container to suit its viscosity and intended use by the consumer. For example, a lotion or fluid cream can be packaged in a bottle or a roll-ball applicator or a propellant-driven aerosol device or a container fitted with a pump suitable for finger operation. When the composition is a cream, it can simply be stored in a non-deformable bottle or squeeze container, such as a tube or a lidded jar. The invention accordingly also provides a closed container containing a cosmetically acceptable composition as herein defined.

While the present invention has been described herein with some specificity, and with reference to certain preferred embodiments thereof, those of ordinary skill in the art will recognize numerous variations, modifications and substitutions of that which has been described which can be made, and which are within the scope and spirit of the invention. It is intended that all of these modifications and variations be within the scope of the present invention as described and claimed herein, and that the inventions be limited only by the scope of the claims which follow, and that such claims be interpreted as broadly as is reasonable. Throughout this application, various publications have been cited. The entireties of each of these publications are hereby incorporated by reference herein.

What is claimed is:

1. A personal care method of modulating MAP2K2 and/or DUSP1 genes in skin of an individual comprising applying to the skin an active ingredient for conditioning of the skin.

2. A personal care method of conditioning photo-damaged skin comprising inhibiting downregulation or upregulating MAP2K2 and /or DUSP-1 genes by topically applying to the skin an active ingredient therefor.

3. The method of claim 1, wherein said modulation is selected from the group consisting of inhibiting downregulation and upregulating.

4. A personal care method of identifying active ingredients for conditioning of skin comprising:
a) using MAP2K2 and/or DUSP-1 gene as a marker of photo-damage in skin;
b) applying an ingredient to the skin of an individual or to epidermal cells; and
c) determining a change in the marker to determine efficacy of the ingredient for treating photo-damage.

5. The method of claim 4, wherein said change in said marker comprises modulation.

6. The method of claim 4, wherein said modulation comprises inhibiting downregulation or upregulating MAP2K2 and /or DUSP-1 genes in said skin.

7. A personal care skin conditioning composition comprising:
a) an active ingredient identified using the method of claim 6; and
b) a cosmetically acceptable vehicle.

8. A personal care method for identifying a photo-damaged skin condition in an epidermal sample, the method comprising the steps of:
a) using MAP2K2 and/or DUSP-1 genes or proteins encoded thereby as markers of photo-damage; and
b) detecting a change in the marker to determine presence of photo-damage.

9. The method of claim 1 wherein said skin condition is selected from the group consisting of photo-damage, aging, appearance of wrinkles, age spots, stratum corneum flexibility, dry skin, acne, oily skin, general quality of skin, skin desquamation and epidermal differentiation.

10. The method of claim 1 wherein said skin condition is photo-damage.

11. The method of claim 8, wherein said gene is MAP2K2;
and wherein said change in said marker is downregulation when said epidermal sample is that of chronically sun-damaged skin.

12. The method of claim 1, wherein said MAP2K2 and/or DUSP1 include proteins encoded thereby.

13. The method of claim 8 comprising a first step of selecting a first skin sample that is known not to be photo-damaged; a nest step of selecting a second skin sample the condition of which is to be identified; comparing said first sample to said second sample to determine whether there is a change in the gene or the amount of the gene, or the protein encoded thereby.
